(19) United States
(12) Patent Application Publication
(10) Pub. No.: US 2014/0088032 A1
(43) Pub. Date: Mar. 27, 2014

(54) TREATMENT OF MASTITIS

(71) Applicants: Thomas Simon Ilg, Nieder-Olm (DE); Ralf Warrass, Worrstadt (DE); Peter Gerardus Franciscus Cox, Bouchemaine (FR); Carlos De Sa, Angers (FR); Karine Pere, Beaucouze Cedex (FR)

(72) Inventors: Thomas Simon Ilg, Nieder-Olm (DE); Ralf Warrass, Worrstadt (DE); Peter Gerardus Franciscus Cox, Bouchemaine (FR); Carlos De Sa, Angers (FR); Karine Pere, Beaucouze Cedex (FR)

(21) Appl. No.: 14/116,910

(22) PCT Filed: May 11, 2012
(86) PCT No.: PCT/EP2012/058712

§ 371 Date: Nov. 11, 2013

Related U.S. Application Data
(60) Provisional application No. 61/485,304, filed on May 12, 2011.

(30) Foreign Application Priority Data
May 12, 2011 (EP) 11165858.9

(51) Int. Cl. A61K 31/546; A61K 31/7036; A61K 31/43; A61K 31/739

(52) U.S. Cl. CPC A61K 31/546; A61K 31/739; A61K 31/7036; A61K 31/43
USPC 514/39; 514/61

(57) ABSTRACT

The invention relates to a veterinary composition for intramammary administration comprising a combination of an antibacterial agent and a Toll-Like-Receptor (TLR) agonist and to the use of said composition for the treatment of mastitis in lactating ruminants.

The present invention relates to veterinary compositions for intramammary administration and to the use of said composition for the treatment of mastitis in lactating cows.

Bovine mastitis is an inflammation of the mammary gland primarily caused by bacterial intramammary infection. Mastitis is the single most costly disease in dairy cows, that causes damage exceeding two billion $ in the USA alone. This disease has a high prevalence (up to 50%) and is caused by a variety of gram-positive bacteria (such as Staphylococcus (S.) aureus or Streptococcus (Str.) uberis), or gram-negative bacteria (such as Escherichia (E.) coli or Klebsiella (K.) pneumoniae) and Mycoplasma (such as M. bovis). In antibiotics treatment of bovine mastitis, cure rates are good (>90%) for infections with most bacteria, with the exception of S. aureus infections, where cure rates are low, in the range of 20-50% (Barkema et al. “The role of cow, pathogen and treatment regimen in the therapeutic success of bovine S. aureus mastitis” J. Dairy Sci. 89, 1877-1895, 2006).

There are a number of hypotheses that seek to explain the high persistency of S. aureus infections in the bovine udder. They include biofilm formation, sequestration of bacteria into intracellular compartments and pathogen-dependent or pathogen-specific induction of inflammatory mediators leading to inadequate help from the innate immune system in the case of S. aureus. Particularly the difference between E. coli mastitis, with acute rapid inflammation, but also usually rapid clearance of infection, and S. aureus mastitis, with much slower, weaker and different inflammatory response, but high tendency to become chronic, is striking (Bannerman et al. “E. coli and S. aureus elicit differential innate immune responses following intramammary infection” Clin. Diagn. Lab. Immunol. 11, 463-472, 2004; Petzl et al. “E. coli, but not S. aureus triggers an early increased expression of factors contributing to the innate immune defence in the udder of the cow.” Vet. Res. 39, 18, 2008).

There have been a multitude of studies published suggesting that much of the pronounced inflammatory response in E. coli udder infections is due to the presence of lipopolysaccharide (LPS), a complex glycolipid of the outer membrane of gram-negative bacteria, but absent in S. aureus. In fact, by LPS challenge of udder quarters, much of the E. coli mastitis symptoms can be mimicked (Shuster et al. J. Dairy Sci. “Suppression of milk production during endotoxin-induced mastitis.” 74, 3763-3774, 1991).

LPS is an agonist (activator) of TLR-4, one member of the Toll-like receptors (TLR) which are involved in pathogen recognition and the evocation of inflammatory responses. Agonists of TLR have shown promise in eliciting non-specific protection against a wide variety of pathogens.

It has been early on recognized, that the induction of an inflammatory response in the bovine udder by LPS could be beneficial for preventing or curing bacterial mastitis caused by homologous or heterologous germs (Kauf et al., Research in Veterinary Science; “Effect of intramammary infusion of bacterial lipopolysaccharide on experimentally induced Staphylococcal aureus mastitis.” 82, 39-46, 2007).

However these previous studies have focussed on stand-alone LPS treatment for prevention or cure of e.g. S. aureus infection. The reported results have not been positive enough (e.g. transient effects only, efficacy too low) to lead to a commercial follow-up.

It has been further recognized that infectious diseases can be treated by initiating qualitative and quantitative changes in the phagocytic cells of the treated animals by a phagocytic-cell activating agent, e.g. a combination of immune modulators, antimicrobials and chemo attractants (EP 405 315).

There remains a need for more effective methods for the treatment of bacterial induced mastitis, especially S. aureus mastitis. To this end the current invention provides a composition for intramammary administration to a mammal, comprising a combination of one or more antibacterial agents and Toll Like Receptor (TLR) agonists as sole active ingredients.

It has been found that the treatment of bacterial induced mastitis, especially S. aureus mastitis, by a veterinary composition of the invention results in higher bacteriological and clinical cure rates in comparison with veterinary compositions comprising the antibacterial agent as the sole therapeutic agent.

Mastitis can be caused by a broad spectrum of bacteria, including gram-positive, gram-negative and wall-less (e.g. Mycoplasma bovis) bacteria. Staphylococcus aureus, coagulase-negative staphylococci and Streptococcus uberis are among the most prevalent gram-positive bacteria to cause the disease. Other gram-positive pathogens are Streptococcus agalactiae, Streptococcus dysgalactiae and Enterococcus spp. Among the gram-negative bacteria that cause mastitis. Escherichia coli, Klebsiella pneumonia, Citrobacter spp., Pseudomonas aeruginosa, Serratia marcescens and Enterobacter aerogenes are the most common.

In order to form the veterinary composition according to the invention the antibacterial agent and the TLR agonist may be present in the dosage form as true mixtures, but they may also be administered individually in separate dosage forms and form mixtures only when they are in the udder.

In case the antibacterial agent and the TLR agonist are administered individually in separate dosage forms, they are administered in parallel. Parallel means that the antibacterial agent and the TLR agonist may be administered at the same time to the udder quarter, that is simultaneously, but they may also be administered sequentially, that is one after the other i.e. so that they are present together for certain periods in the affected udder quarter, so that the desired effect arises.

In one embodiment the antibacterial agent and the TLR agonist are administered simultaneously. When given simultaneously the composition according to the invention is preferably presented as a single dosage form comprising both the antibacterial agent and the TLR agonist in a single formulation.

The antibacterial agent that is included in the compositions of the invention can be in general an antibacterial agent or a combination of antibacterial agents with sufficient broad spectrum antibacterial efficacy in order to treat the most important micro-organisms causing mastitis. Such antibacterial agents are generally known in the art, such as the β-lactam antibiotics, the aminoglycoside antibiotics, the macrolide antibiotics, the tetracycline antibiotics and others.

Preferred is a β-lactam antibiotic, e.g. a penicillin or cephalosporin. In a preferred embodiment it is a cephalosporin. Cephalosporins are semisynthetic antibiotics derived from cephalosporin C, a natural antibiotic produced by the mould Cephalosporium acremonium. Cephalosporins belong to the class of β-lactam antibiotics and are classified as first-(e.g. cephapirin, cephalothin, cephaloridine, cefazolin), second-(cefamandole, cefuroxime, cefoxitin), third-(e.g. cefotaxime, ceftriaxone, cefoperazone, ceftiofur) or fourth-generation (cefepime, cefpirome, cefquinome) products according to their spectrum of activity and the position and type of side-chain that has been incorporated into the basic molecule. At present cephalosporins are widely used for the treatment of infections.

The term “cephalosporins” when used herein includes pharmaceutically acceptable salts and esters thereof.

Combination of antibacterial agents that are useful in the compositions of the present invention are for example a β-lactam antibiotic, such as ampicillin or penicillin in combination with an aminoglycoside, such as neomycin or kanamycin. A particular preferred combination is penicillin, especially benzyl penicillin with neomycin.

A particular preferred cephalosporin compound is cephapirin. Cephapirin (3-[(acetoxy)methyl]-8-oxo-7[[4-pyridinylthio)acetyl]amino]-5-thia-1-azabicyclo-[4.2.0]oct-2-ene-2-carboxylic acid) is a cephalosporin of the first generation. Preferably the pharmaceutically acceptable salt of cephapirin is the sodium salt.

In another preferred embodiment of the invention the fourth-generation cephalosporin cefquinome (I1-[[(6R,7R)-7-[[(2Z)-(2-amino-4-thiazolyl)-(methoxyimino)acetyl]amino]-2-carboxy-8-oxo-5-thia-1-azabicyclo[4.2.0-oct-2-en-3-yl]methyl]-5,6,7,8-tetrahydroquinolinium inner salt) is used. Cefquinome is a semi-synthetic aminothiazolyl cephalosporin resembling cefotaxime. Preferably the pharmaceutically acceptable salt of cefquinome is the sulphate salt.

A typical composition according to the invention comprises 10 to 500 mg of the antibacterial agent depending on the potency of the compound. Preferably the veterinary composition comprises 200 to 400 mg of cephapirin/unit dose, more preferred 300 mg.

In a preferred embodiment a veterinary composition of the invention for use in lactating cows comprises 3 doses of 50 to 150 mg of cefquinome/unit dose, more preferred 50 to 100 mg/unit dose, specifically preferred 75 mg/unit dose, each unit dose being applied with a 12 hours interval (at 3 consecutive milkings).

The Toll-Like-Receptor agonist that is included in the composition of the invention can be any ligand that is recognized by one of the family of TLRs and induces an immunostimulant response.

TLRs are critical for the innate immune response in mammals. TLRs are transmembrane glycoproteins, which are expressed in leukocytes and the epithelial cells of mucosal surfaces, and which are composed of extracellular, trans membrane and intracellular signalling domains. The extracellular domains have leucine-rich repeat modules and are responsible for binding distinct ligands that are broadly shared by pathogens, collectively known as pathogen-associated molecular patterns (PAMPs). Upon binding with said ligands activation of the TLR signalling pathway occurs, the initial step being the ligand-induced dimerization of TLRs on the cell membrane (Jin and Lee; “Structures of Toll-like Receptor Family and its Ligand Complexes”, Immunity, 29, 182-191, 2008). There are two major types of TLRs, those, i.e. TLR3, TLR7, TLR8 and TLR9, that reside in intracellular compartments (in the membrane of endosomes) and which can be activated by viral and bacterial nucleic acids, and those, i.e. TLR1, TLR2, TLR4, TLR5 and TLR6, that are expressed on the cell surface and which can be activated by outer membrane components of bacteria, fungi and protozoan organisms. Upon TLR activation by an agonistic compound a signalling pathway is triggered culminating in the production of pro-inflammatory mediators, such as chemokine, cytokines and cell adhesion molecules (Kaiwa and Akira; “Signaling to NF-k8 by Toll-like receptors” Trend Mol. Med. 13, 460-469, 2007).

TLR agonists/ligands that can be used in the compositions of the present invention are the lipoproteins or lipopeptide derivatives recognized by TLR2 in complex with TLR1 or TLR6, such as the synthetic triacylated lipopeptide PAM₃CSK₄ (Jin and Lee; supra), that retains most of the immune stimulatory activity of the full-length lipoproteins, diacylated lipoprotein derivatives, such as fibroblast-stimulating lipopeptide-1 (FSL-1), i.e. Pam₂CGDPKHPKSF, derived from Mycoplasma salivarium, and macrophage-activating lipopeptide-2 (MALP-2) from M. fermentans, i.e. S-[2,3-bispalmitoyloxy-(2R)-propyl]-cysteinyl-SNNDESNISFKEK, yeast zymosan (betaglucan) and lipoteichoic acid.

TLR agonists recognized by TLR3 are viral or synthetic double stranded RNA preparations, such as poly I/C or poly A/U.

TLR4 agonists for use in the veterinary compositions of the invention are lipopolysaccharide preparations from natural or mutant strains of gram-negative bacteria, especially the hydrophobic component thereof referred to as lipid A (Wang and Quinn; ‘Lipopolysaccharides: biosynthetic pathway and structure modification.”, Progress in Lipid Research 49, 97-107, 2010) and synthetic derivatives thereof (Gaekwad et al., ‘Differential induction of innate immune response by synthetic lipid A derivatives”, J. Biol. Chem. 285, 29375-29386, 2010), such as lipid A or Kdo-lipid A from N. meningitides and lipid A or Kdo₂-lipid A from E. coli. A preferred TLR4 polysaccharide chain agonist for use in the composition of the invention is LPS or Kdo₂-lipid A (Re-LPS) of E. coli. The advantage of Kdo2-Lipid A over LPS is that it is a reproducible in its production and a more defined natural product, and it can be detected by ESI/MS at the low concentrations used to stimulate animal cells.

Lipopolysaccharide (LPS) agonists with short polysaccharide chains (rough-LPS, usually found on mutant strains) are classified as chemotypes Ra, Rb, Rc, Rd and Re, wherein the subscript designate the polysaccharide length of a given LPS. Ra-LPS and Re-LPS designate the mutants with the longest and shortest chain length, respectively.

TLR agonists recognized by TLR5 are the flagellin proteins from flagellated gram-positive and gram-negative bacteria.

TLR agonists recognized by TRL7 and TLR8 are single-stranded RNA and synthetic small molecule agonists, such as imidazoquinolines, like R848, imiquimod and thiazoloquinoline, like CL075.

TLR agonists recognized by TRL9 are microbial DNAs or synthetic oligonucleotides derived thereof, preferentially phosphorothioates and phosphodiester oligonucleotides.

There is a preference for the use of TLR agonists that activate the cell-surface located TLR1, TLR2, TLR4, TLR5 or TLR6, such as exemplified by Re-LPS, PAM₃CSK₄, FSL-1, flagellin and zymosan.

Identification of further Toll-like Receptor agonists for use in the compositions of the invention can for instance be done with the use of a NF-κB-Luciferase or secreted alkaline phosphatase gene reporter assay in HEK293-bovineTLR transfectants. Such transfectants may carry any one of the bovine TLR receptors, preferably bovTLR4, bovTLR5, bovTLR3, bovTLR7 and bovTLR8. Such process is also described in our co-pending European Patent application No. 10197435.0, incorporated by reference herein.

Methods to determine receptor binding as well as in vitro biological activity of TLR modulators are well known in the art. In general, expressed receptor is contacted with the compound to be tested and binding or stimulation or inhibition of a functional response is measured.

To measure a functional response isolated DNA encoding the TLR receptor gene, preferably the bovine receptor, is expressed in suitable host cells, such as a HEK293 cell.

Methods to construct recombinant cell lines are well known in the art (Sambrook et al, Molecular Cloning: a Laboratory Manual, Cold Spring Harbor Laboratory Press, Cold Spring Harbor, latest edition). Expression of the receptor is attained by expression of the DNA encoding the desired protein. Techniques for ligation of additional sequences and construction of suitable expression systems are all, by now, well known in the art. Portions, or all of the DNA encoding the desired protein can be constructed synthetically using standard solid phase techniques, preferably to include restriction sites for ease of ligation. Suitable control elements for transcription and translation of the included coding sequence can be provided to the DNA coding sequences. As is well known, expression systems are now available which are compatible with a wide variety of hosts, including prokaryotic hosts such as bacteria and eukaryotic hosts such as yeast, plant cells, insect cells, mammalian cells, avian cells and the like.

Cells expressing the TLR receptor are then contacted with the test compound to observe binding, or stimulation or inhibition of a functional response.

Functional TLR receptor agonist activity may be measured by determining the modulation of signaling cascades, such as for example measurement of receptor mediated changes in API, NFκB or IRF transcription factor activations. Thus, such a method involves expression of the TLR receptor on the cell surface of a host cell and exposing the cell to the test compound. The production of induced transcripts or derived protein products (e.g. interleukin-8) is then measured. The level of transcript or derived protein will be reduced or increased, depending on the effect of the test compound upon binding to the receptor.

In addition to direct measurement of induced transcript or protein in the exposed cell, cells can be used which in addition to transfection with receptor encoding DNA are also transfected with a second DNA encoding a reporter gene, the expression of which correlates with receptor activation. In general, reporter gene expression might be controlled by any response element reacting to changing levels of second messenger. Suitable reporter genes are e.g. LacZ, alkaline phosphatase, firefly luciferase and green fluorescence protein. The principles of such transactivation assays are well known in the art and are described e.g. in Stratowa, C. A. et al., Curr. Opin. Biotechnol. 6, 574 (1995).

A useful cell line for the detection of TLR4 agonists is a HEK293 cell transfected with a bovine TLR4 plasmid, with plasmids containing the potentiating bovine MD2 and bovine CD14 sequences, and with a NFκB reporter gene construct, which senses the activation by a TLR4 agonist.

A useful cell line for the detection of agonists for TLR1, TLR2 or TLR6 agonists is a bovine macrophage cell line (BOMAC) that expresses these receptors and transfected with bovine CD14, in order to potentiate the response to lipoprotein derived agonists, and with a NFκB reporter gene construct, which senses the activation by the TLR agonist.

A useful cell line for the detection of TLR3, TLR5, TLR7 and TLR8 agonists is a HEK293 cell line transfected with one of said bovine TLR plasmids and with a NFκB reporter gene construct, which senses the activation by the TLR agonist.

For selecting active agonist compounds on the respective TLR receptor the EC₅₀ value must be <10⁻⁵ M, preferably <10⁻⁷ M, more preferred <10⁻⁸ M.

A preferred composition according to the invention comprises an antibiotic agent and an agonist of TLR4. A preferred TLR4 agonist is a lipopolysaccharide, especially a derivative of lipid A, such as Re-LPS (Kdo2-lipid A).

A particular preferred veterinary composition of the invention comprises the antibiotic cefquinome in combination with the TLR4 agonist Re-LPS (Kdo2-lipid A).

Another particular preferred veterinary composition of the invention comprises the antibiotics penicillin and neomycin in combination with the TLR4 agonist Re-LPS (Kdo2-lipid A).

A typical composition according to the invention comprises an amount of a TLR agonist that is depending on the potency of the compound. In a preferred veterinary composition of the invention for use in lactating cows wherein the TLR4 agonist is a LPS derivative, such as re-LPS, the composition comprises 1-5, especially 3 doses of 1-20 μg/unit dose, more preferred 1-5, especially 3 doses of 10 μg (or 1500 EU)/unit dose. In one embodiment each unit dose is applied with a (approximate) 12 hours interval between the administrations (at 1-5, especially 3 consecutive milkings).

The treatment schedule comprises 1 to 5, especially 3 consecutive administrations of the antibacterial agent and the TLR4 agonist during each milking.

Because the potency to cause pyrogenic reaction varies according to the nature of an LPS preparation, the potency of a LPS (endotoxin) derivative is preferably expressed in Endotoxin Units/mg (EU/mg). These units were developed by the FDA, as well as the assays for their determination. The most commonly used approach is an assay based on the reaction of an LPS derivative with Limulus Amoebocyte Lysate (LAL-test), an aqueous extract of blood cells from the horseshoe crab (see European Pharmacopeia 7.0; Chapter 2.6.14. Bacterial Endotoxins.), in comparison with a known standard (100 pg of the US standard Endotoxin EC-5 has the activity of 1 EU). A preferred veterinary composition of the invention for the treatment of mastitis in lactating cows, comprises 500-3000 EU of LPS derivative for administration per udder quarter, preferably 1500 EU per quarter.

The composition of the invention may further comprise pharmaceutically acceptable auxiliaries, such as a carrier. The pharmaceutically acceptable carrier for the active ingredients (the antibacterial agent and the TLR agonist) is selected so as to be non-toxic, pharmaceutically acceptable, compatible with the active ingredients, and of a viscosity to permit administration, whilst controlling the release characteristics of the drug particles.

In accordance with common practice, the veterinary composition according to the invention for intramammary administration comprises a suspension or solution of the active ingredient in a suitable carrier, which can be made of an aqueous or oily base. In one embodiment the carrier is an oily base. The antibiotic component and the TLR agonist component of the veterinary compositions of the invention may be formulated together in a single pharmaceutical formulation or may be formulated in two separate pharmaceutical formulations.

Oils that can be used for the oily base in veterinary compositions are in general natural, e.g. vegetable, semi-synthetic or synthetic mono-, di- or tri glyceride. Vegetable oils that can be used are e.g. sesame oil, olive oil, cottonseed oil, castor oil, arachis oil, or coconut oil. The pharmaceutically acceptable carrier in the composition according to the invention preferably comprises an oily base and optionally comprises one or more additives such as thickening agents, desiccants and antioxidants. Suitable pharmaceutical excipients are known in the art. Such pharmaceutical excipients for the carrier for intramammary formulations are e.g. described in “Gennaro, Remington: The Science and Practice of Pharmacy” (20th Edition, 2000), incorporated by reference herein.

Conventional thickening agents are e.g. aluminum stearate, silica, or fatty acid esters such as glycerol monostearate. A suitable amount of a thickening agent is within the range of 0 to 30% by weight. Desiccants are e.g. silicates, activated clay, silica gel, and molecular sieve. Especially preferred is sodium aluminum silicate. A suitable amount of a desiccant that can be used is within the range of 0 to 15% by weight, preferably 0-10%. Suitable antioxidants are e.g. butylhydroxytoluene or hydroxyanisole. The antioxidant will usually be present within the range of 0 to 10% by weight. Other additives may also be present in the carrier in minor proportions.

The invention provides furthermore a veterinary composition for use in therapy, and especially for use in the treatment of mastitis in lactating cows.

Veterinary compositions of the invention are especially useful in the treatment of bacterial induced mastitis, especially mastitis caused by Staphylococcus spp. or Steptococcus spp. pathogens, especially by Staphylococcus aureus, Staphylococcus epidermidis, Steptococcus agalactiae or Steptococcus uberis infection.

The veterinary composition according to the invention can be applied principally to all non-human mammalian species that need treatment of clinical mastitis. Mastitis may affect any mammalian species, but is especially important in ruminants that are used for milk production for human consumption such as cattle, buffalo, camelids, sheep and goats.

Another aspect of the invention is a kit useful in the treatment of a mastitis, especially mastitis caused by S. aureus infection in a non-human mammal, which comprises a unit dose of an antibacterial agent in a veterinary acceptable formulation and a unit dose of a TLR agonist agent in a veterinary acceptable formulation and instructions for their parallel intramammary administration.

As used herein, “a,” an” and “the” include singular and plural referents unless the context clearly dictates otherwise. Thus, for example, reference to “an active ingredient” includes a single active ingredient as well as two or more different active ingredients in combination, reference to “a carrier” includes mixtures of two or more carriers as well as a single carrier, and the like.

### EXPERIMENTAL

### Example 1

### Materials

S. aureus

A Staphylococcus aureus strain Newbould 305 (ATCC 29740) stock suspension (about 10¹¹ CFU/ml) was kept frozen at −80° C. in peptone physiological water with 10% of glycerol. On day 0, the stock solution was thawed and diluted in peptone physiological water to a concentration of ca 1000 cfu/ml.

### Cefquinome Sulphate

The injector for intramammary use contains 75 mg of the antibiotic cefquinome per 8 gram in an oily phase. The MIC of cefquinome against the S. aureus strain used for the challenge Newbould 305 is 0.25 μg/ml.

### Re-LPS.

Re-LPS, also called Kdo₂-lipid A, is a preparation of the saccharolipid glycan extracted from the cell wall of E. coli. (Raetz, et al. “Kdo2-Lipid A of Escherichia coli, a defined endotoxin that activates macrophages via TLR-4.” J Lipid Res 47, 1097-111, 2006). The product was obtained from Avanti Polar Lipids, USA; batch nr.: KDOO2LA-12) as a 1 μg/ml solution in sterile water and has the following nearly homogenous Re-LPS substructure with endotoxin activity equal to that of native LPS:

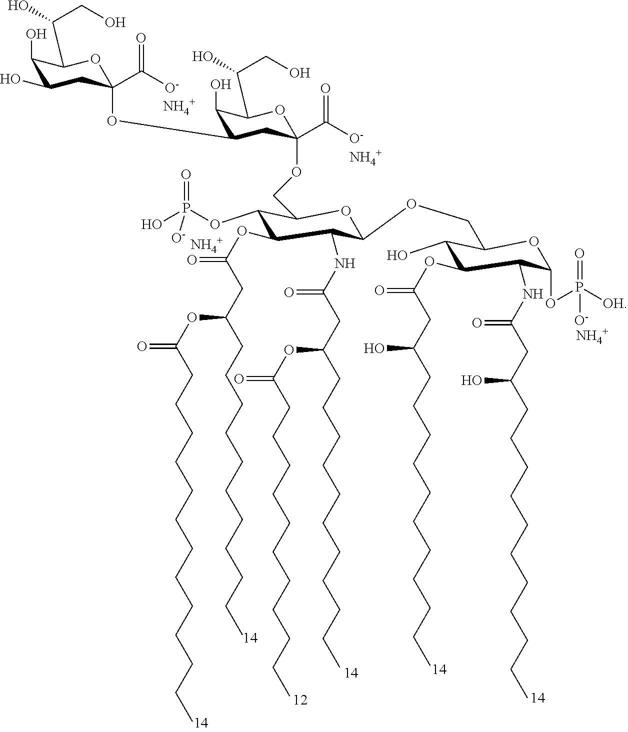

Kdo2-Lipid A is an intermediate in the synthesis of LPS. It has two 3-deoxy-D-manno-octulosonic acid (Kdo) sugar residues in place of the core, and has no O-antigen. Its ability to activate TLR4 is comparable to that of native LPS.

### Test System:

On Day 0 8 Prim'Holstein lactating cows, animals of between 2-5 years old and at least 2 months in lactation, were intramammarily inoculated into two homolateral quarters with 1 ml of S. aureus suspension per quarter (ca. 1000 cfu/quarter) into the teat cistern with sterile 2-ml syringes. Teat ends were disinfected with towels soaked in a disinfectant solution before infusion.

On day 6 after the challenge 4 cows (8 quarters) were treated with 75 mg cefquinome—one injector after each of 3 consecutive milkings—Group A. The 4 other cows infected were treated with 75 mg cefquinome as well, but 10 μg of re-LPS were infused just before each cefquinome infusion—Group B.

The standard assay for measuring mastitis in the dairy industry is to count the number of cells (originating from the cow) in the milk. This estimate is termed the somatic cell count (SCC). Specifically, the SCC reflects the levels of cells, including immune cells, such as leukocytes that are released from the lining and tissues of the udder of the infected animal, into the udder cavity. The number of somatic cells in a given volume of milk (typically 1 ml) provides a semi-quantitative estimate of the degree of mastitis, because unaffected animals typically have low SCC levels.

Milk samples were investigated for S. aureus presence and SCC levels at several time points during the study (days 2, 6, 9, 13, 16, 20, 23 post challenge for S. aureus bacteriology).

Bacteriological cure was assessed 16 days after treatment end (i.e. 23 days after the challenge) and was defined as having at least the two last consecutive milk samples found negative for S. aureus (<10 cfu/ml) with acceptable SCC levels (<250 000 cells/ml).

Clinical cure was assessed 16 days after treatment end (i.e. 23 days after the challenge), and was defined as having at least the two last consecutive milk samples with SCC <250000 cells/ml.

Summary of the results are presented in Table 1 below.

Numbers indicated are the SCC; lettering type indicates S. aureus bacteriological status (normal lettering=negative sample, bold/underlined lettering=S. aureus positive sample)

TABLE 1
Quarter milk SCC (×10³ cell/ml) at day relative to infection (day 0)

| Group | Quarter | −8 | 0 | 2 | 6 | 9 | 13 | 16 | 20 | 23 | Cure |
|---|---|---|---|---|---|---|---|---|---|---|---|
| A | RF | 78 | 231 | 4665 | 876 | 533 | 414 | 317 | 589 | 186 | yes |
| | RR | 44 | 70 | 3581 | 542 | 763 | 250 | 74 | 137 | 98 | yes |
| A | RF | 13 | 13 | 2388 | 1615 | 334 | 177 | 125 | 177 | 109 | yes |
| | RR | 12 | 2 | 1908 | 24 | 795 | 112 | 785 | 1686 | 2482 | no |
| A | RF | 42 | 29 | 5594 | 601 | 249 | 134 | 95 | 77 | 47 | yes |
| | RR | 58 | 60 | 9999 | 2980 | 628 | 384 | 2032 | 2008 | 1382 | no |
| A | RF | 76 | 77 | 887 | 542 | 310 | 828 | 921 | 717 | 739 | no |
| | RR | 46 | 52 | 2597 | 227 | 521 | 1533 | 254 | 281 | 536 | no |
| B | LF | 11 | 11 | 5117 | 1579 | 1080 | 169 | 81 | 35 | 24 | yes |
| | LR | 8 | 18 | 9485 | 1292 | 1142 | 166 | 70 | 83 | 21 | yes |
| B | RF | 12 | 9 | 808 | 258 | 2463 | 345 | 117 | 90 | 58 | yes |
| | RR | 13 | 8 | 733 | 297 | 3110 | 188 | 48 | 30 | 18 | yes |
| B | RF | 4 | 2 | 3669 | 309 | 4691 | 423 | 138 | 25 | 20 | yes |
| | RR | 5 | 7 | 9586 | 551 | 2998 | 469 | 9999 | 892 | 1211 | no |
| B | RF | 3 | 5 | 983 | 235 | 2127 | 222 | 44 | 25 | 16 | yes |
| | RR | 205 | 161 | 140 | 337 | 1958 | 332 | 103 | 56 | 58 | excl |

Infection with S. aureus at Day 0

Treatment at day 6 with cefquinome (group A) or cefquinome+re-LPS (group B)

Following infection a marked increase of SCC was generally observed.

Re-LPS treatment induced SCC levels above 1.10⁶ cells per ml. From day 13, SCC values were comparable in both groups. Quarters considered as cured at day 23 had generally normal SCC levels already from days 13 or 16.

At the end of the study (16 days after treatment end), bacteriological cure rates were as follows: 50% for cefquinome (4 quarters out of 8), and 86% for re-LPS/cefquinome group (6 quarters out of 7, 1 quarter was excluded because of S. aureus negative status following infection).

TABLE 2

| | Quarters cure rates at day 23 | | |
|---|---|---|---|
| | | | bacteriological + |
| | bacteriological | clinical | clinical |
| Group A | 50.0% (4/8) | 37.5% (3/8) | 37.5% (3/8) |
| Group B | 85.7% (6/7) | 85.7% (6/7) | 85.7% (6/7) |

### Example 2

### Materials

S. aureus

A Staphylococcus aureus strain Newbould 305 (ATCC 29740) stock suspension (about 10⁹ CFU/ml) was kept frozen at −80° C. in peptone physiological water with 10% of glycerol. On day 0, the stock solution was thawed and diluted in peptone physiological water to a concentration of ca 200 cfu/ml.

### Neomycin+Penicillin Treatment

Neomycin and Benzyl penicillin were formulated as a single oily suspension containing the 2 antibiotics. The formulation was filled in 10-mL single use injectors for intramammary treatment. Each injector contained 250 mg neomycin and 169 mg benzylpenicillin per 8 grams of oily base. MICs of neomycin and benzyl penicillin against the S. aureus Newbould strain used for the challenge are 2 μg/ml and 0.064 μg/ml, respectively.

### Re-LPS

Re-LPS, also called Kdo₂-lipid A, is a preparation of the saccharolipid glycan extracted from the cell wall of E. coli. (Raetz, et al. “Kdo2-Lipid A of Escherichia coli, a defined endotoxin that activates macrophages via TLR-4.” J Lipid Res 47, 1097-111, 2006). The product was obtained from Avanti Polar Lipids, USA; it is a nearly homogenous Re-LPS substructure with endotoxin activity comparable to that of native LPS:

Kdo2-Lipid A is an intermediate in the synthesis of LPS. It has two 3-deoxy-D-manno-octulosonic acid (Kdo) sugar residues in place of the core, and has no O-antigen. Its ability to activate TLR4 is comparable to that of native LPS.

Re-LPS was prepared as a 1 μg/ml solution in sterile water.

### Test System:

On Day 0, 12 Prim'Holstein lactating cows, animals of between 2-8 years old and at least 1 month in lactation, were inoculated by intramammary route into two homo-lateral quarters, with 5 ml of S. aureus suspension per quarter (ca. 1000 cfu/quarter), using 5-ml syringes. Teat ends were disinfected with towels soaked in a disinfectant solution before infusion.

On day 7 after the challenge, infected quarters from 6 cows (12 quarters) were treated with neomycin/penicillin (group A). Treatment consisted in the infusion of 1 neomycin/penicillin injector per infected quarter. Two infusions were performed in total, with an interval of 24 h between each infusion. The 6 other cows infected were treated with neomycin/penicillin injectors as well, but 10 μg of re-LPS were infused just before each neomycin/penicillin infusion—Group B.

The standard assay for measuring mastitis in the dairy industry is to count the number of cells (originating from the cow) in the milk. This estimate is termed the somatic cell count (SCC). Specifically, the SCC reflects the levels of cells, including immune cells, such as leukocytes that are released from the lining and tissues of the udder of the infected animal, into the udder cavity. The number of somatic cells in a given volume of milk (typically 1 ml) provides a semi-quantitative estimate of the degree of mastitis, because unaffected animals typically have low SCC levels.

Milk samples were investigated for S. aureus presence and SCC levels at several time points during the study (days 3, 7, 10, 14, 17, 21, 24 post challenge for S. aureus bacteriology).

Bacteriological cure was assessed 16 days after treatment end (i.e. 24 days after the challenge) and was defined as having at least the two last consecutive milk samples found negative for S. aureus (<5 cfu/ml).

Clinical cure was assessed 16 days after treatment end (i.e. 24 days after the challenge), and was defined as having at least the two last consecutive milk samples with SCC <250000 cells/ml.

Summary of the results are presented in Table 3 below.

TABLE 3
Quarter milk SCC (×10³ cell/ml) at day relative to infection (day 0)

| | | | | | | | | | | | Bacterio. |
|---|---|---|---|---|---|---|---|---|---|---|---|
| group | quarter | −6 | 0 | 3 | 7 | 10 | 14 | 17 | 21 | 24 | cure |
| A | RF | 2 | 4 | 280 | 294 | 347 | 24 | 12 | 5 | 4 | yes |
| | RR | 2 | 6 | 2977 | 257 | 243 | 23 | 13 | 6 | 7 | yes |
| A | RF | — | — | — | — | — | — | — | — | — | excl. (1) |
| | RR | 12 | 29 | 9999 | 590 | 1607 | 93 | 35 | 25 | 18 | yes |
| A | RF | 91 | 82 | 6994 | 1410 | 896 | 3822 | 4978 | 9999 | 9999 | no |
| | RR | 18 | 22 | 9999 | 2391 | 2276 | 8291 | 9760 | 2710 | 2474 | no |
| A | RF | 13 | 6 | 7266 | 585 | 2437 | 77 | 38 | 29 | 27 | yes |
| | RR | 10 | 6 | 72 | 15 | 1427 | 43 | 14 | 4 | 5 | excl. (2) |
| A | RF | 3 | 1 | 1274 | 410 | 257 | 53 | 37 | 16 | 12 | yes |
| | RR | 5 | 4 | 839 | 389 | 342 | 35 | 33 | 7 | 7 | yes |
| A | RF | 23 | 62 | 1258 | 288 | 773 | 115 | 147 | 98 | 85 | yes |
| | RR | 27 | 114 | 379 | 404 | 909 | 208 | 1961 | 1873 | 804 | no |
| B | RF | 5 | 3 | 6176 | nd | 5064 | 120 | 30 | 27 | 30 | yes |
| | RR | 8 | 6 | 7586 | nd | 5182 | 160 | 54 | 23 | 17 | yes |
| B | RF | 5 | 5 | 672 | 649 | 3086 | 4566 | 1130 | 417 | 252 | yes |
| | RR | 39 | 30 | 449 | 978 | 2220 | 168 | 59 | 6639 | 763 | no |
| B | RF | 5 | 8 | 4913 | 1399 | 1062 | 316 | 73 | 153 | 30 | yes |
| | RR | 4 | 108 | 4063 | 1826 | 2814 | 91 | 72 | 46 | 32 | excl. (2) |
| B | RF | 31 | 88 | 244 | 98 | 3745 | 385 | 310 | 87 | 82 | yes |
| | RR | 28 | 103 | 2387 | 452 | 3574 | 241 | 129 | 69 | 126 | yes |
| B | RF | 20 | 28 | 219 | 779 | 1455 | 80 | 50 | 34 | 19 | yes |
| | RR | 30 | 31 | 1989 | 526 | 2275 | 93 | 54 | 27 | 21 | yes |
| B | RF | 6 | 6 | 6856 | 563 | 3985 | 269 | 172 | 38 | 25 | yes |
| | RR | 4 | 6 | 9999 | 1563 | 5314 | 227 | 90 | 33 | 30 | yes |

Numbers indicated are the SCC; lettering type indicates S. aureus bacteriological status (normal lettering = negative sample, bold/underlined lettering = S. aureus positive sample)

Infection with S. aureus at day 0. Treatment at day 7 with neomycin/penicillin (group A) or neomycin/penicillin+re-LPS (group B).

Following infection a marked increase of SCC was generally observed. Exclusions: (1) quarter not fitting with inclusion criteria; (2) quarters where S. aureus was not recovered before treatment (after challenge).

Re-LPS treatment induced SCC levels above 1.10⁶ cells per ml. From day 14, SCC values were comparable in both groups. Quarters considered as cured at day 24 had generally normal SCC levels already from days 14 or 17.

At the end of the study (16 days after treatment end), bacteriological cure rates were as follows: 70% for neomycin/penicillin (7 quarters out of 10, 2 quarters were excluded), and 91% for reLPS-neomycin/penicillin group (10 quarters out of 11, 1 quarter was excluded).

Cure rates are summarized in table 4 below:

TABLE 4

| | Quarters cure rates at day 24 | | |
|---|---|---|---|
| | | | bacteriological + |
| | bacteriological | clinical | clinical |
| Group A | 70% (7/10) | 70% | 70% (7/10) |
| Group B | 91% (10/11) | 82% (9/11) | 82% (9/11) |

### Example 3

A Streptococcus uberis strain (099/0024) will be used to induce mastitis in lactating cows. After challenge, animals will be treated with neomycin/penicillin or neomycin/penicillin combined with re-LPS.

### Preparation of the Inoculum.

The Str. uberis inoculum for the intramammary challenge will be prepared in a physiological medium at a concentration of about 200 cfu/ml.

### Neomycin+Penicillin Treatment

Neomycin and Benzyl penicillin will be formulated as indicated above in Example 2.

### Re-LPS.

Re-LPS will be prepared as indicated above in Example 1.

### Study Design:

On Day 0, 10 Prim'Holstein lactating cows, animals of between 2-8 years old and at least 1 month in lactation, will be inoculated by intramammary route into two homolateral quarters, with 5 ml of Str. uberis suspension per quarter (ca. 1000 cfu/quarter).

On day 3 after the challenge, infected quarters from 5 cows (10 quarters) will be treated with neomycin/penicillin (group A). Treatment will consist in the infusion of 1 neomycin/penicillin injector per infected quarter. Two infusions will be performed in total, with an interval of 24 h between each infusion. The 5 other cows infected (group B) will be treated with neomycin/penicillin injectors as well, but 10 μg of re-LPS will be infused just before each neomycin/penicillin infusion.

Milk samples will be investigated for Str. uberis presence and Somatic Cell Counts (SCC) levels at several time points during the study (days 3, 6, 10, 13, 17, and 24 post challenge for Str. uberis bacteriology).

Bacteriological cure will be defined as having at least the two last consecutive milk samples found negative for Str. uberis.

Clinical cure will be defined as having at least the two last consecutive milk samples with SCC <250000 cells/ml.

Cure rates will be compared between the 2 treatments.

1. A veterinary composition for intramammary administration comprising a combination of active ingredients wherein the active ingredients consist of an antibacterial agent and a Toll-Like-Receptor (TLR) agonist.

2. (canceled)

3. The composition of claim 1, wherein the TLR agonist is a lipopolysaccharide.

4. The composition of claim 3, wherein the lipopolysaccharide is a lipid A derivative.

5. The composition of claim 4, wherein the lipid A derivative is Kdo2-lipid A.

6. The composition of claim 1, wherein the antibacterial agent is a β-lactam antibiotic.

7. The composition of claim 6, wherein the β-lactam antibiotic is a penicillin.

8. The composition of claim 6, wherein the composition comprises a β-lactam antibiotic and neomycin.

9. The composition of claim 1 wherein the antibacterial agent is cefquinome or a pharmaceutically acceptable salt thereof.

10. The composition of claim 1, wherein the antibacterial agent is cefquinome and the TLR agonist is Kdo2-lipid A.

11. The composition of claim 1, wherein the antibacterial agent is penicillin and neomycin and the TLR agonist is Kdo2-lipid A.

12. (canceled)

13. A method of treating mastitis in ruminants comprising administering to the ruminant an effective amount of the composition of claim 1.

14. A kit for the treatment of mastitis in a non-human mammal which consists of a unit dose of an antibacterial agent in a veterinary acceptable formulation and a unit dose of an TLR agonist agent in a veterinary acceptable formulation and instructions for their parallel intramammary administration.

15. The composition of claim 1, wherein the antibacterial agent is a aminoglycoside antibiotic.

16. A method of treating mastitis in ruminants comprising administering to the ruminant an effective amount of the composition of claim 10.

17. A method of treating mastitis in ruminants comprising administering to the ruminant an effective amount of the composition of claim 11.

18. A method of treating mastitis in ruminants comprising administering to the ruminant an effective amount of the composition of claim 15.
